(19) United States
(12) Patent Application Publication
(10) Pub. No.: US 2019/0390399 A1
(43) Pub. Date: Dec. 26, 2019

(54) FUNGAL COMPOSITES COMPRISING MYCELIUM AND AN EMBEDDED MATERIAL

(71) Applicant: Mycoworks, Inc., Emeryville, CA (US)

(72) Inventors: Jordan Chase, El Cerrito, CA (US); Philip Ross, San Francisco, CA (US); Nicholas Wenner, Sebastopol, CA (US); William Morris, San Francisco, CA (US)

(21) Appl. No.: 16/453,791

(22) Filed: Jun. 26, 2019

Related U.S. Application Data
(60) Provisional application No. 62/690,101, filed on Jun. 26, 2018.

(51) Int. Cl. D06M 16/00; C12N 1/14

(52) U.S. Cl. CPC D06M 16/003; D10B 2331/04; D10B 2401/063; C12N 1/14

(57) ABSTRACT

A flexible fungal composite with an engineered and/or improved mechanical properties such as tear strength, tensile strength and resistance to separation. The fungal composite is generated by embedding a second material within a fungal matrix. The tear strength of the fungal composite is greater than the tear strength of the fungal matrix. The tensile strength of the fungal composite is at least equal to the tensile strength of the embedded material. And the resistance to delamination between the fungal matrix and the embedded material is such that the force required to separate the fungal matrix and the embedded material from each other is greater than or equal to the force required to separate the fungal matrix or the embedded material from themselves.

### RELATED APPLICATIONS

This application claims the benefit of U.S. provisional patent application 62/690101, filed Jun. 26, 2018, the disclosure of which is incorporated herein in its entirety

### BACKGROUND OF THE DISCLOSURE

### Technical Field of the Disclosure

The present embodiment relates generally to fungal composites, and more particularly, to a fungal composite exhibiting enhanced properties, with engineered degrees of symmetry, isotropy versus anisotropy, and localized zones having distinct characteristics.

### Description of the Related Art

Bio-composite materials are widely utilized in construction, the automotive industry, bio-medical engineering and in various other engineering applications. Main constituents of the bio-composite materials are biopolymers and bio-based reinforcing agents. Bio-composite materials have certain advantages over petroleum-based products because of their improved fuel efficiency, environmental friendliness, renewability, biodegradability, and low cost.

A nascent family of bio-composite materials is enabled by mycelium. Mycelium is the vegetative component of a fungus or fungal colony, and it is commonly utilized as a bio-based, renewable, and biodegradable matrix within various bio-composite materials. Mycelium based bio-composites can be easily achieved via inoculation of agricultural waste; such a method is commonly used for growing fungal fruiting bodies (mushrooms) for general human consumption, culinary use, as well as for other industrial purposes. In most commercial cases, the bio-composite resulting from the inoculation and colonization of agricultural waste using fungal mycelium, is merely a byproduct that is not engineered as a bespoke material.

The process of colonization is the phenomenon of the fungal colony growing, whereby the mycelial network permeates and penetrates each and every particle of its nutritional media: the agricultural waste. As the mycelium envelopes its food, it forms new connections thereto in order to facilitate its metabolic process and thereby break down the food source on and throughout which it lives. The amount of food energy the mycelia can absorb is directly proportional to how much media is interfaced with and adhered to.

Adhesion is a key property of mycelium that enables it to adhere to any other materials it comes in contact with, via extensions of its most basic, morphological components: hyphae. Hyphae are the discrete units (e.g. arms, branches, etc.) of a mycelial network. Each hypha can exude the enzymes that result in metabolism of food or defense against foreign organisms or chemicals. From a macroscopic perspective, a mycelial mass, being a network of hyphae, appears as a fluffy, soft mass of spongy material. Microscopically, the vegetative mycelium that comprises the majority of a single fungal colony further comprises a network of branched, filamentous hyphae. This network of hyphae grows and propagates via self-extension from any given strand, as well as by branching and splitting and reconnecting throughout any substrate or media it is inoculated with.

As shown in FIG. 1A, a macroscopic view of a prior art existing mycelium mass is illustrated. FIG. 1B shows a prior art microscopic view of a small region of the mycelium mass shown in FIG. 1A, wherein the white horizontal line shows the length of 100 μm. The hyphal network is clearly distinguishable. The small filaments each constitute an individual hypha. FIGS. 1A and 1B illustrate adhesive, interconnected properties and the morphological nature of the mycelium that can make up one component of a fungal composite.

In nature, a fungal colony will expand via the growth of its mycelia within a volume of soil, within a dead tree, or even into free space at the interface of solid media and a surrounding environment. The individual hyphae are small enough in diameter that they may spread throughout tiny, interstitial spaces while still remaining invisible to the naked eye. If two, discrete mycelium masses are placed in contact; the mycelium spreads amongst them and effectively bonds them together. Fungal mycelia are perfectly capable of self-cannibalizing, which can result in strong adhesion between adjacent mycelial masses. This self-adhesive or cohesive property of mycelium enables the fungal tissues to produce bio-composites adaptable to form different hard and solid configurations.

Several methods have been developed for producing fungus-based bio-composite materials. One such method describes a mechanism for culturing filamentous fungi specifically for the production of materials and composites composed in part, or entirely of, hyphae and its aggregate form, mycelia and mycelium. The composite material is made by inoculating a substrate of discrete particles and a nutrient material with a preselected fungus. Even so, this method implements complex methodologies and hardware. The state of the art of such a process also produces rigid objects that exhibit nearly zero flexibility, as well as limited strength and elongation.

Another method describes a living, hydrated mycelium composite containing at least one of a combination of mycelium and fibers, mycelium and particles, and mycelium, particles and fibers. The mycelium living composite is dehydrated and then rapidly re-formed into many different shapes, such as bricks, blocks and pellets. However, the generated mycelium composite exhibits low flexibility and high brittleness.

Yet another method describes steps for growing a fungus polymer matrix that is composed predominately of fungal tissues. The resultant material is a flexible and soft, high-density amorphous polymer that can serve in applications that are currently served by synthetic plastics and foams, as well as some instances wherein animal skins are deployed. This method for generating fungus polymer matrix is expensive, and the means for producing materials has a high environmental impact. The resulting bio-composites based on this art have little to no utility as a material that requires tensile and tear strength to compete with textiles and animal leathers. Furthermore, the state of the art requires long time periods of fabrication and post-processing that are incompatible with large-scale production.

In each of the described instances of related art, the bio-composite is only formed between a fungal organism (the mycelial mass), other fungal tissues, and/or the fungal organism's food supply. The interaction between the organism and its food may generically be referred to as fermentation: the conversion of matter from one form into another, using a living organism. In each of the aforementioned cases, agricultural waste is converted from its raw form into mycelium via the process of the mycelium growing and generating more of itself.

Therefore, there is a need for a mycelium-based bio-composite that integrates a mycelium matrix and a second material other than its primary nutritional media. Similarly, there is a need for an efficient and reliable method for generating fungal composites having high self-adhesive property as well as enhanced and engineerable mechanical properties. Such a needed method would produce an improved fungal composite utilizing simple techniques and hardware. Further, such a generated fungal composite would exhibit high flexibility and high tensile strength. Moreover, such a method for generating an improved fungal composite would be cost effective and would have a low environmental impact. The present embodiment accomplishes these and other objectives.

### SUMMARY OF THE INVENTION

To minimize the limitations found in the prior art, and to minimize other limitations that will be apparent upon the reading of the specification, the preferred embodiment of the present invention provides a fungal composite for improved strength, flexibility, flexural life, elongation, adhesion, cohesion, resistance to separation, colorfastness, abrasion, and softness. Furthermore, the fungal composite may be engineered to exhibit completely isotropic properties, a pre-selected degree of both isotropy versus anisotropy, completely orthotropic properties, or a combination thereof. Finally, the fungal composite may include localized zones or regions exhibiting distinct properties relative to the global properties of the entire composite.

In a preferred embodiment, the fungal composite comprises a fungal matrix and an embedded material adaptable to being embedded within the fungal matrix in order to generate the fungal composite. The tear strength of the fungal composite is greater than the tear strength of the fungal matrix. The tensile strength of the fungal composite is at least equal to the tensile strength of the embedded material, or greater than either material alone. The resistance to delamination can be implemented between the fungal matrix and the embedded material such that the force required to separate the fungal matrix and the embedded material from each other is greater than or equal to the force required to separate the fungal matrix and the embedded material from themselves.

Preferably, the fungal matrix is a mycelium matrix. The fungal composite achieves the improved strength through space-filling of the fungal matrix within the embedded material as well as through physical and chemical linking of the fungal matrix and the embedded material through various methods. The embedded material includes, but is not limited to, cotton, silk, wool, polyester, polyamide; or other materials comprising synthetics such as plastic, semi-synthetics such as rayon or viscose, and natural or organic materials such as cellulose. The embedded material may be singly or in any combination of the aforementioned list. It may be of any structure, including but not limited to knit or woven, felt, open-cell foam, singly or in any combination, and as such may be solid or liquid phase at the time of embedding and integration with the fungal matrix. Materials and structures additional to the embedded material may also be embedded within the fungal matrix.

A first objective of the present invention is to provide a fungal composite having improved mechanical properties that are engineered, controlled, and of greater commercial value than either of the two combined materials alone.

A second objective of the present invention is to provide a fungal composite having improved tear strength, tensile strength, resistance to delamination, flexibility, flexural life, colorfastness, abrasion resistance, and softness.

A third objective of the present invention is to provide a fungal composite with high self-adhesive property, adhesion, cohesion, resistance to separation, and resistance to delamination.

A fourth objective of the present invention is to provide a fungal composite with an ability to stretch or elongate to a degree that is greater than either any the combined materials alone.

Another objective of the present invention is to provide a fungal composite utilizing a cost effective method and hardware.

Another objective of the present invention is to provide a fungal composite having low environmental impact.

Another objective of the present invention is to provide a fungal composite having high stitch-tear strength.

Another objective of the present invention is to provide a fungal composite having the ability to be constructed as animal free, petroleum and plastic free, vegetable-based, or other configurations commensurate with being vegan, vegetable, organic, etc.

Another objective of the present invention is to provide a fungal composite that can be bio-degraded faster than other mechanically equivalent materials

These and other advantages and features of the present invention are described with specificity so as to make the present invention understandable to one of ordinary skill in the art.

### BRIEF DESCRIPTION OF THE DRAWINGS

In order to enhance their clarity and improve understanding of these various elements and embodiments of the invention, elements in the figures have not necessarily been drawn to scale. Furthermore, elements that are known to be common and well understood to those in the industry are not depicted in order to provide a clear view of the various embodiments of the invention. Thus, in the interest of clarity and conciseness, the drawings are generalized in form.

FIG. 1A shows a photograph of a macroscopic view of an existing type of mycelium mass;

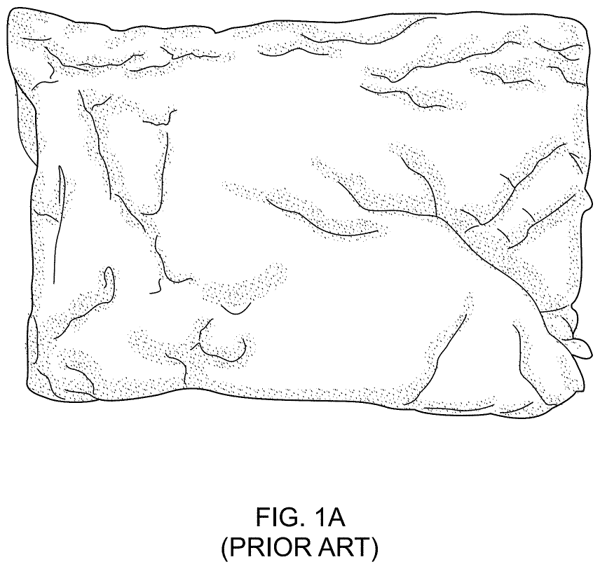

FIG. 1B shows a photograph of a microscopic view of a region of the mycelial mass shown in FIG. 1A, illustrating an existing hyphal network;

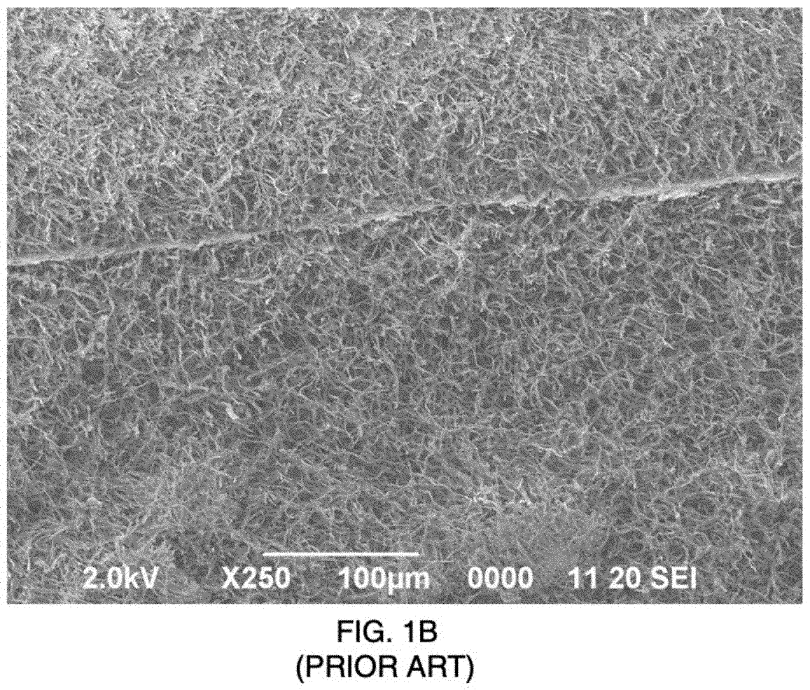

FIG. 2 shows tensile testing samples of polyester felt and fungal leather/polyester felt composite materials according to the preferred embodiment of the present invention;

FIG. 3 is a chart of results from tensile testing of polyester felt and fungal material/polyester felt composite materials illustrated in FIG. 2 according to the preferred embodiment of the present invention;

FIG. 4 shows a bar graph representing a quantitative comparison of strength and flexibility of mycelium and a potential embedded textile according to the preferred embodiment of the present invention; and

FIG. 5 shows a photograph of another embodiment of the present invention, illustrating the space filling properties of the mycelium matrix according to the preferred embodiment of the present invention.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

As used herein, the singular forms “a”, “an” and “the” include plural referents unless the context clearly dictates otherwise. “And” as used herein is interchangeably used with “or” unless expressly stated otherwise. As used herein, the term “about” means +/−5% of the recited parameter. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

The present embodiment is a fungus-based composite material having engineered mechanical properties relevant to the commercial use of the specific product. Table 1 is a summary of demonstrable performance properties that are achieved through tailored design of the one embodiment of the fungus-based composite.

TABLE 1

| Property | Range | Unit |
|---|---|---|
| Thickness | 0.6-4.0 | [mm] |
| Specific Weight | 600-1250 | [g/m²] |
| Tensile Force | 20-50 | [kg/cm] |
| Ultimate Tensile Strength | 5-50 | [Mpa] |
| Elongation at break: MIN-MAX | 20-150 | [%] |
| Tongue Tear | 2.5-15 | [kg] |
| Stitch Tear Strength | 0.1-10 | [kg/cm] |
| Mullen Burst | 30 | [kg/cm²] |
| Bally Flex (cycles) | 1-250,000 | [cycles] |
| Stoll Abrasion | 1-3,000 | [cycles at 1 lbf] |
| Martindale Abrasion | 1-10,000 | [cycles] |
| Softness (Smooth Haircell) | 1-5 | [mm/hour] |
| Softness (Floater) | 1-5 | [mm/hour] |
| Drape Coefficient | 1-100 | [%] |
| Colorfastness, Hydrolysis | 3.5-5 | [AATCC 8 Scale] |
| (Crocking) | | |
| Colorfastness, Distilled Water | 3.5-5 | [AATCC 8 Scale] |
| Colorfastness, Wicking Solvent | 3.5-5 | [AATCC 8 Scale] |
| Colorfastness, Wash Test | 3.5-5 | [AATCC 8 Scale] |
| Finish Adhesion | 2-5 | [kg/cm] |
| Ply Adhesion | 1-5 | [kg/cm] |

The enhanced mechanical properties are preferably between the low and high end of the ranges in each property row above in Table 1. For each range, the low end can in some cases be considered as a minimum with no upper bound, and the high end can further be considered a maximum with no lower bound.

The fungal composite comprises a fungal matrix and an embedded material adaptable to embed within the fungal matrix to generate the fungal composite. The tear strength of the fungal composite is greater than the tear strength of the fungal matrix. The tensile strength of the fungal composite is at least equal to the tensile strength of the embedded material. The resistance to delamination or any other form of separation, through chain entanglement or any suitable mechanism between the fungal matrix and the embedded material is such that the force required to separate the fungal matrix and the embedded material from each other is greater than or equal to the force required to separate the fungal matrix or the embedded material from themselves. The resistance to delamination can be measured using standard testing methods such as ASTM D2724: Standard Test Methods for Bonded, Fused, and Laminated Apparel Fabrics.

In the preferred embodiment, the fungal matrix is a mycelium matrix. The embedded material includes, but is not limited to, cotton, silk, polyester, polyamide, wool, rayon, nylon, Dyneema, viscose and cellulose, singly or in any combination. It can be of any structure, including but not limited to knit, woven, felt, or open-cell foam, singly or in any combination. Materials and structures additional to the embedded material may also be embedded within the fungal matrix.

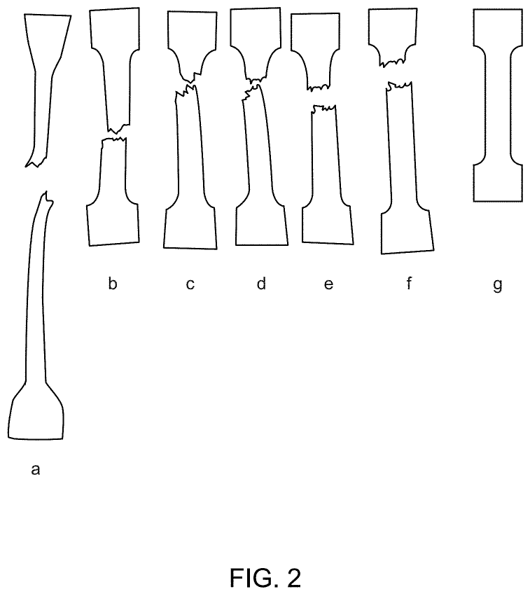

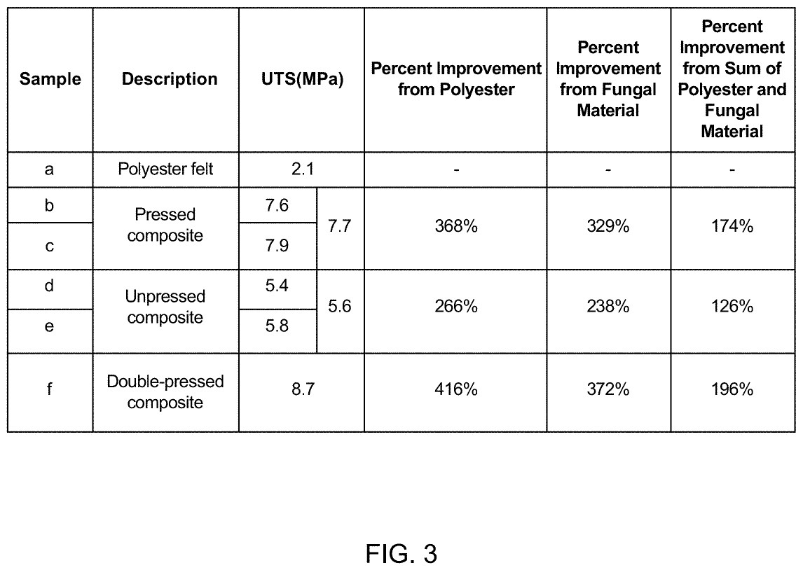

In one preferred embodiment of the present invention, the fungal matrix is combined or embedded with a polyester felt as shown in FIG. 2. Here, tensile testing samples of polyester felt denoted by the letter ‘a’ and fungal leather/polyester felt composite materials denoted by the letters ‘c’, ‘d’, ‘e’ and ‘f’ are illustrated. And ‘g’ denotes the fungal matrix. In this case, the ultimate tensile strength of the resulting material is greater than the sum of the tensile strengths of the constituent materials. The result of this tensile test is illustrated in FIG. 3.

TABLE 2

| Material | UTS Lower [Mpa] |
|---|---|
| Polyester felt | 2.1 |
| Pure fungal | 2.4 |
| material | |
| TOTAL | 4.5 |

Referring to Table 2, the ultimate tensile strengths of polyester felt and pure fungal material are detailed. This Table 2 merely illustrates the superposition of the individual strengths of the mycelium and a polyester felt embedded material. The composite materials of FIG. 3 show tensile strengths that are 1.3×-2.0× the sum of the ultimate tensile strengths of the constituent materials that are superimposed in Table 2.

FIG. 3 shows a chart of results from tensile testing of polyester felt and fungal material/polyester felt composite materials illustrated in FIG. 2. The composite materials are unpressed, pressed, or double-pressed referring to a process by which the samples are permanently compressed with the application of heat and pressure. Even the unpressed composite samples exhibit greater tensile strength than the sum of the tensile strengths of their constituent parts.

The fungal composite achieves the improved strength through space-filling of the fungal matrix within the matrix of the polyester felt as well as through physical and chemical linking of the fungal matrix and the embedded material through various methods including but not limited to chain entanglement, penetration of fungal hyphae into the polyester fibers, surface adhesion of the fungal hyphae onto the surfaces of the polyester fibers and any other suitable mechanisms. The mutual reinforcement of the fungal matrix and the embedded material prevents fibers from aligning toward parallel and slipping past each other, as may happen when either of the two materials is pulled in tension on their own. This is shown in FIG. 2, where the non-reinforced polyester felt sample ‘a’, extended greatly under tension as its fibers aligned toward parallel before failing through the separation between the fibers.

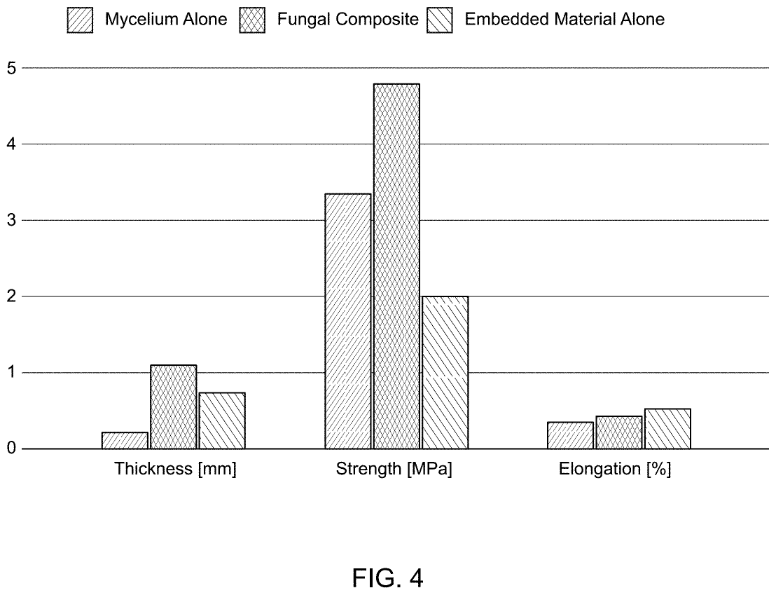

FIG. 4 shows a bar graph representing a quantitative comparison of thickness, strength and flexibility (elongation) of mycelium, of a potential embedded textile, as well as of a bio-composite comprising mycelium and a potential embedded textile. In this case, strength is measured in MPa and flexibility is measured in percent elongation. FIG. 4 illustrates graphically that the strength of the bio-composite of mycelium plus an embedded material is stronger than the separate materials alone. Similarly, the elongation measured for the bio-composite falls numerically between the separate materials on their own, conveying a mechanical marriage of the two.

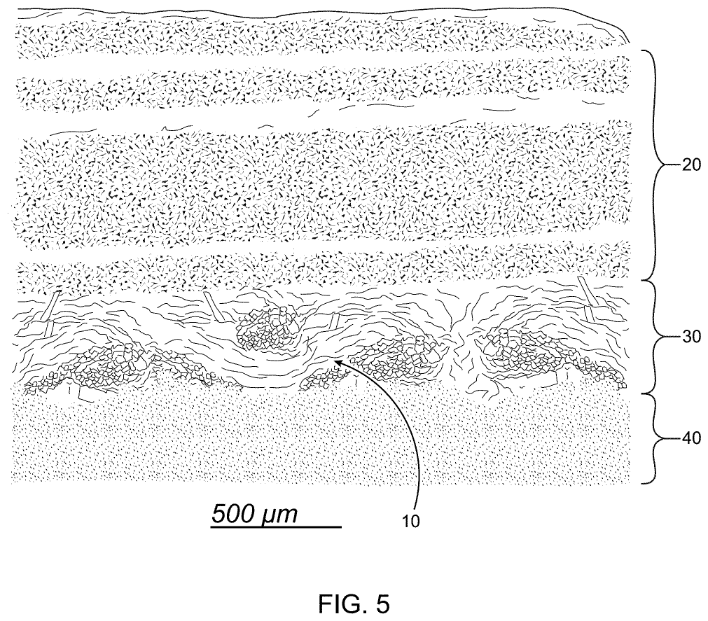

FIG. 5 is a high-magnification image of another embodiment of the present invention illustrating a fungus-based bio-composite, wherein the black line shows the length of 500 μm. Referring to FIG. 5, the space filling properties of the mycelium matrix in an embedded material is shown. In this embodiment, the entanglement of the network of hyphae comprising the mycelial matrix, as well as the mycelium penetrating and space-filling within the interstitial spaces throughout the embedded material layer is illustrated. Here, a woven material 10 is utilized as the embedded material within the mycelium matrix. According to FIG. 5, mycelium grows in three layers: a first layer 20, a second layer 30 and a third layer 40. That is, the mycelium grows through and above the woven textile 10 in the first layer 20, grows within and throughout the woven textile 10 in the second layer 30 and grows below the woven textile 10 in the third layer 40.

In alternative embodiments the embedded material may include one or more of a number of various materials. The embedded material may be vegetable based, protein based, animal derived, plastic derived, organic, etc., in order to engineer a composite material that is vegan, organic, biodegradable, non-biodegradable, or animal-free. In certain other embodiments the embedded material is one or more of cotton, silk, wool, rayon, polyester, polyamide, viscose, or cellulose. In certain other embodiments the embedded material is integrated in a liquid phase or state, or the embedded material is added in a liquid phase, and chemically reacted to generate a viscous, semi-viscous, or solid phase of the same material, or chemically reacted complex derived from the original, embedded material.

In practice, the composite exhibits isotropic properties, anisotropic properties, orthotropic properties, or a combination thereof in key or specific areas. The composite may comprise an engineered construction or layout of embedded materials such that the composite exhibits specifically engineered properties in pre-selected areas or regions or zones of the composite when considered as a singular whole. The composite exhibits equivalent, equal, and symmetric properties throughout the entire composite when considered as a singular whole. The composite preferably comprises a plurality of regions, wherein each region has distinct characteristics due to having a distinct construction of embedded materials in each region.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. A composite comprising:
a) a fungal matrix having a set of fungal matrix mechanical properties; and
b) an embedded material within the fungal matrix, the combination making up a fungal composite, and the embedded material having a set of embedded material mechanical properties;
c) whereby the fungal composite exhibits a set of fungal composite mechanical properties that is greater than either the fungal matrix mechanical properties or embedded material mechanical properties alone, and wherein the mechanical properties comprise tear strength, tensile strength, flexural strength, resistance to separation and resistance to delamination; and
d) wherein the elongation of the composite falls numerically or quantitatively between the elongation of the mycelium matrix alone and the embedded material alone.

2. The composite of claim 1 wherein the embedded material is one of cotton, silk, wool, manufactured fiber, nylon, polyester, polyamide, viscose, or cellulose.

3. The composite of claim 1 wherein the composite exhibits isotropic properties, anisotropic properties, orthotropic properties, or a combination thereof in key or specific areas.

4. The composite of claim 1 wherein the composite comprises an engineered construction or layout of embedded materials such that the composite exhibits specifically engineered properties in pre-selected areas or regions or zones of the composite when considered as a whole.

5. The composite of claim 1 wherein the composite comprises equivalent, equal, and symmetric properties throughout the entire composite when considered as a whole.

6. The composite of claim 1 wherein the composite comprises a plurality of regions, wherein each region has distinct characteristics due to having a distinct construction of embedded materials in each region.

7. The composite of claim 1 wherein the embedded material is integrated in a liquid phase or state.

8. The composite of claim 1 wherein the embedded material is added in a liquid phase, and chemically reacted to generate a viscous, semi-viscous, or solid phase of the embedded material, or chemically reacted complex derived from the original, embedded material.

9. The composite of claim 1 wherein the fungal matrix is a mycelium matrix.

10. A composite comprising:
a) a fungal matrix; and
b) an embedded material selected from the group consisting of cotton, silk, polyester, wool, manufactured fiber, viscose, or cellulose, wherein the embedded material is embedded within the fungal matrix, the combination making up a fungal composite;
wherein the tear strength of the fungal composite is greater than the tear strength of the fungal matrix;
wherein the tensile strength of the fungal composite is greater than the tensile strength of the embedded material; and
wherein the resistance to delamination between the fungal matrix and the embedded material is such that the force required to separate the fungal matrix and the embedded material from each other is greater than or equal to the force required to separate the fungal matrix or the embedded material from themselves.

11. The composite of claim 10 wherein the tear strength of the fungal composite is greater than or equal to the tear strength of the fungal matrix.

12. The composite of claim 10 wherein the tensile strength of the fungal composite is greater than or equal to the tensile strength of the embedded material.

13. The composite of claim 10 wherein the resistance to delamination between the fungal matrix and the embedded material is such that the force required to separate the fungal matrix and the embedded material from each other is greater than the force required to separate the fungal matrix or the embedded material from themselves.

14. The composite of claim 10 wherein the fungal matrix is a mycelium matrix.

15. The composite of claim 10 wherein the mechanical properties are selected from the group consisting of tear strength, tensile strength, flexural strength, elongation, resistance to separation or resistance to delamination.

16. The composite of claim 10 wherein the fungal composite achieves the improved strength through physical and chemical linking of the fungal matrix and the embedded material through methods including but not limited to chain entanglement, penetration of fungal hyphae into the embedded material, surface adhesion of the fungal hyphae onto the surfaces of the embedded material, colonization of the embedded material by the hyphal network, and/or integration and cohesion of the hyphal network to the embedded material though growth phenomenon related to metabolic processes of the hyphae within a nutritional substrate.

17. A composite comprising:
a) a fungal matrix; and
b) an embedded material within the fungal matrix, the combination making up a fungal composite;
wherein the tear strength of the fungal composite is greater than or equal to the tear strength of the fungal matrix;
wherein the tensile strength of the fungal composite is greater than or equal to the tensile strength of the embedded material; and
wherein the resistance to delamination between the fungal matrix and the embedded material is such that the force required to separate the fungal matrix and the embedded material from each other is greater than the force required to separate the fungal matrix or the embedded material from themselves.

18. The composite of claim 17 wherein the embedded material is one of cotton, silk, wool, manufactured fiber, nylon, polyester, polyamide, viscose or cellulose.

19. The composite of claim 17 wherein the elongation of the composite falls numerically or quantitatively between the elongation of the mycelium matrix alone and the embedded material alone.

20. The composite of claim 17 wherein the composite comprises a plurality of regions, wherein each region has distinct characteristics due to having a distinct construction of embedded materials in each region.

21. The composite of claim 17 wherein the tear strength and tensile strength of the fungal composite is greater than the tear strength of the fungal matrix.
